(19) United States
(12) Patent Application Publication
(10) Pub. No.: US 2013/0180339 A1
(43) Pub. Date: Jul. 18, 2013

(54) PRESSURE SENSING METHODS, DEVICES, AND SYSTEMS

(71) Applicant: James M. Brugger, Newburyport, MA (US)

(72) Inventor: James M. Brugger, Newburyport, MA (US)

(73) Assignee: NxStage Medical, Inc., Lawrence, MA (US)

(21) Appl. No.: 13/825,310

(22) PCT Filed: Sep. 23, 2011
(86) PCT No.: PCT/US2011/053141

§ 371 Date: Mar. 20, 2013

Related U.S. Application Data
(60) Provisional application No. 61/385,732, filed on Sep. 23, 2010.

(51) Int. Cl. A61B 5/02

(52) U.S. Cl. CPC A61B 5/02
USPC 73/700

(57) ABSTRACT

A pressure measuring device has tubing with an inline “pillow” portion that is more flexible than the tubing portion. The pillow portion may be created by heating a portion of a length of medical tubing and forcing air into the heated portion to expand, and thin, its walls. The tubing and pillow portion may be used in a negatively pressurized portion of a blood circuit such as an arterial return line. An assembly of interleaved support members adhesively bonded to the pillow portion holds the pillow portion in an expanded state even under negative pressure. Further, when the pillow portion tries to collapse, the interleaved support members compress a strain gauge to provide a pressure signal corresponding to the negative pressure exerted by the fluid therewithin.

### CROSS-REFERENCE TO RELATED APPLICATION

This application claims the benefit of priority to U.S. Provisional Patent Application No. 61/385,732 filed Sep. 23, 2010, the entire content of which is hereby incorporated by reference into the present application.

### FIELD

Embodiments relate generally to pressure sensing methods, devices, and systems. In particular, embodiments include systems, methods, and devices that sense or measure negative pressure, in an arterial return line of a fluid circuit, for example.

### BACKGROUND

Various systems, including medical treatment devices, employ pressure monitoring of fluids. There is a need in the art for devices, methods, and system for monitoring negative pressures of fluids in a vessel or channel. In a particular application of note, blood tubing carrying blood from a patient to a treatment device, such as a dialysis system, may be measured using a drip chamber or a variety of other devices. There is a need for improvements in this blood treatment application to reduce costs of medical disposals, prevent blood clotting, and provide high accuracy in measurements.

### SUMMARY

The Summary describes and identifies features of some embodiments. It is presented as a convenient summary of some embodiments, but not all. Further the Summary does not necessarily identify critical or essential features of the embodiments, inventions, or claims.

Disclosed embodiments include a negative pressure measurement device, comprising: a fluid circuit portion for conducting a fluid in a portion of a blood circuit susceptible to negative pressure in support of a blood treatment therapy; said fluid circuit portion having a tubular part with a sensing portion; and a mechanism immediately adjacent the sensing portion of the tubular part that is configured and operative to translate a compliant strain of the fluid circuit portion responsive to a negative pressure therewithin into a force to the sensing portion to generate a signal; wherein the force lies in a different principal direction from a principal direction associated with the strain.

Embodiments also include a pressure measurement device for measuring pressure in a fluid-carrying tube portion of a disposable fluid circuit for a medical treatment system, comprising: a sensor element positioned on support a fluid processing machine; said support being physically coupled to said tube portion when said fluid circuit is mounted on said fluid processing machine; said sensor element being configured to generate a signal in response to change in shape of said tube portion resulting from a negative change in pressure therewithin.

Disclosed embodiments include a pressure measurement or detection method, comprising: detecting a force associated with a negative pressure within a fluid line; generating an electrical signal in response to said detecting; said signal being representative of the negative pressure with the fluid line; the generating including forcing a member attached to a fluid vessel be displaced such that a compression force is applied to a force transducer.

Additionally, embodiments of the disclosed subject matter can include a method of generating a signal corresponding to a pressure in a vessel, comprising: flowing fluid through a flexible vessel to which are attached a pair of members that are mutually movable; subjecting an interior of the flexible vessel to a negative pressure thereby forcing the pair of members to move relative to each other; the subjecting being effective to move at least a portion of one member away from a portion of the other member so as to generate a progressively increasing separation therebetween; generating a pressure signal including measuring a force of the progressively increasing separation.

### BRIEF DESCRIPTION OF THE DRAWINGS

Embodiments will hereinafter be described in detail below with reference to the accompanying drawings, wherein like reference numerals represent like elements. The accompanying drawings have not necessarily been drawn to scale. Any values dimensions illustrated in the accompanying graphs and figures are for illustration purposes only and may not represent actual or preferred values or dimensions. Where applicable, some features may not be illustrated to assist in the description of underlying features.

FIG. 1A is an overhead cross-sectional view of a portion of medical tubing for circulating fluid according to embodiments of the disclosed subject matter.

FIG. 1B is an overhead cross-sectional view of the portion of medical tubing shown in FIG. 1A in a contracted or collapsed state.

FIGS. 2A and 2B are cross-sectional views of a device according to embodiments of the disclosed subject matter, with FIG. 2B being a cross-section view along line A-A.

FIGS. 2C and 2D are select views of portions of the device shown in FIGS. 2A and 2B, with FIG. 2C showing a portion of FIG. 2A and FIG. 2D showing an end view of FIG. 2A only with select portions being viewable.

FIGS. 3A and 3B show a pair of interoperable members that may be adhesively attached to a flexible portion of medical tubing according to embodiments of the disclosed subject matter.

FIG. 4 shows a pressure sensor arrangement with two V-shaped elements and a living hinge according to embodiments of the disclosed subject matter.

FIGS. 5A and 5B show portions of a pressure sensing device as shown in FIG. 5C.

FIGS. 6A through 6D show additional embodiments upon which a basis for pressure sensing devices according to embodiments of the disclosed subject matter are formed.

FIGS. 7A and 7B show two addition embodiments of pressure sensing devices according to the disclosed subject matter.

FIG. 8 shows a diagram of a blood treatment system that can implement pressure sensing devices according to embodiments of the disclosed subject matter.

FIG. 9 is a flow chart for a method according to embodiments of the disclosed subject matter.

### DESCRIPTION

The description set forth below in connection with the appended drawings is intended as a description of various embodiments of the disclosed subject matter and is not intended to represent the only embodiments in which the disclosed subject matter may be practiced. The description includes specific details for the purpose of providing a thorough understanding of the disclosed subject matter. However, it will be apparent to those skilled in the art that the disclosed subject matter may be practiced without these specific details. In some instances, well-known structures and components may be shown in block diagram form in order to avoid obscuring the concepts of the disclosed subject matter.

It can be desirable in extracorporeal blood processing therapies to measure negative pressures in the fluid circuit, such as negative pressures that can be experienced in arterial return lines. It may be further desirous to prevent or minimize internally directed movement or collapsing of relatively more flexible tubing portions at which such negative pressures are measure or sensed.

Generally speaking, embodiments of the disclosed subject matter operate on the principle of turning a negative into a positive. More specifically, since it may be undesirable or impractical to place a pressure sensing device in a blood flow path in order to sense or detect internal negative pressures, embodiments of the disclosed subject matter can translate movement inward of a flexible tube portion due to negative pressure within the tube to movement of a corresponding assembly which can “grow” or otherwise move a portion thereof outward in response to the negative pressure and thereby cause a positive force which can be sensed or detected by a pressure sensing device. Thus, as well as translating the motion this mechanism can transmit the force of the collapsing due to negative pressure and communicate it to a force sensor.

Embodiments can also prevent or lessen movement inward of certain tube portions when negative pressures within the tubing are experienced. Because movement inward is either prevented or lessened of these certain tube portions, embodiments of the disclosed subject matter can also ensure a relatively smooth transition in the flow path when the portion of the tube that is movable moves slightly inward.

Accordingly, embodiments of the disclosed subject matter can hold open the flexible portion of the tube to allow unrestricted flow during negative pressure as well as allow for measurement of the transmitted force which is proportional to the negative pressure.

Embodiments include negative pressure measuring devices having tubing with an inline flexible portion that is more flexible than the tubing portions immediately up-line and down-line thereof. In some cases, this flexible portion may be a “pillow” portion or a thin walled tube portion. For instance, the flexible portion can be thin walled tubing such as NC15-0195 (flexible PVC) and standard patient line tubing such as NC15-0132 (flexible PVC) can be used for the relatively non-flexible tubing.

The tubing and flexible portion may be used in a negatively pressurized portion of a blood circuit such as an arterial return line. In various embodiments, an assembly of support members adhesively bonded to the pillow portion may be provided which can hold the flexible portion in an expanded state even under negative pressure. Further, when the flexible portion tries to collapse, the support members can compress a strain gauge to provide a pressure signal corresponding to the negative pressure exerted by the fluid therewithin.

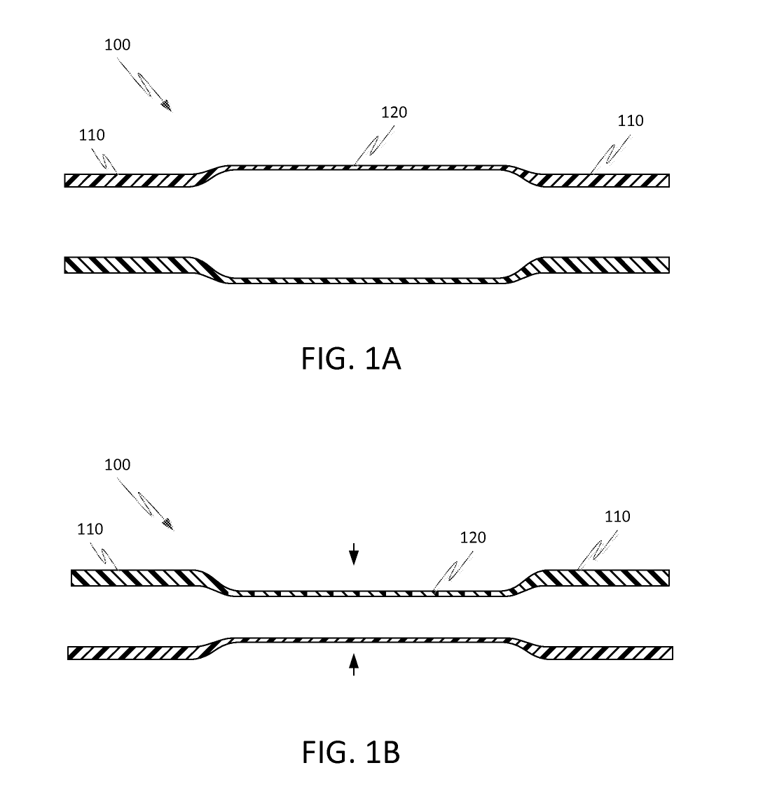

FIG. 1A is a side cross-sectional view of a portion of medical tubing 100 for circulating fluid according to embodiments of the disclosed subject matter. As indicated above, tubing 100 can have a portion that is more flexible than up-line and down-line portions thereof. FIG. 1A shows a so-called pillow portion that is more flexible than surrounding portions 110. The flexible portion, in this example, the pillow portion 120 can be formed or created in any suitable way. For example, starting with a tube of a medical thermoplastic elastomeric material, the tube a portion of a length of medical tubing may be heated and air is then forced into the heated portion or “blown” to expand or stretch, and thereby thin its walls.

However, by itself, the flexible portion, when subjected to negative pressures can contract or collapse (fully or partial), in some cases severely restricting fluid flow. Pressure monitoring also may become more difficult because of such contraction or collapsing. FIG. 1B is a side cross-sectional view of the medical tubing 100 shown in FIG. 1A having its pillow portion 120 in a collapsed state. By adding a structure to the pillow as indicated in the various embodiments set forth herein, the inward collapsing of the medical tube 100 can be translated into outward motion of the structure to thereby sense and measure the negative pressure.

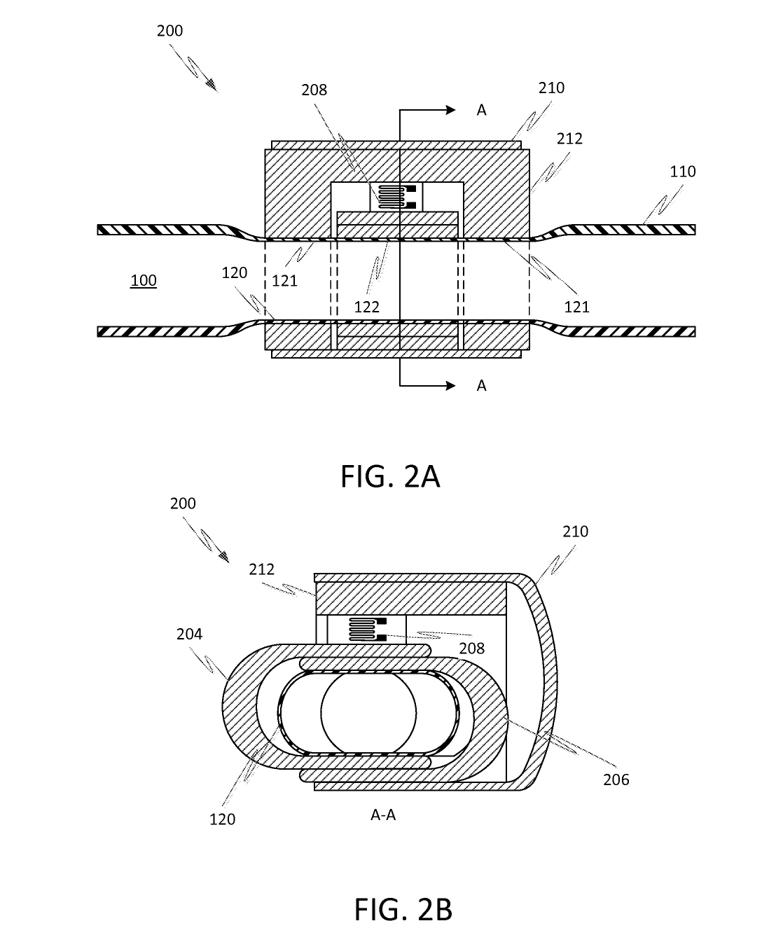

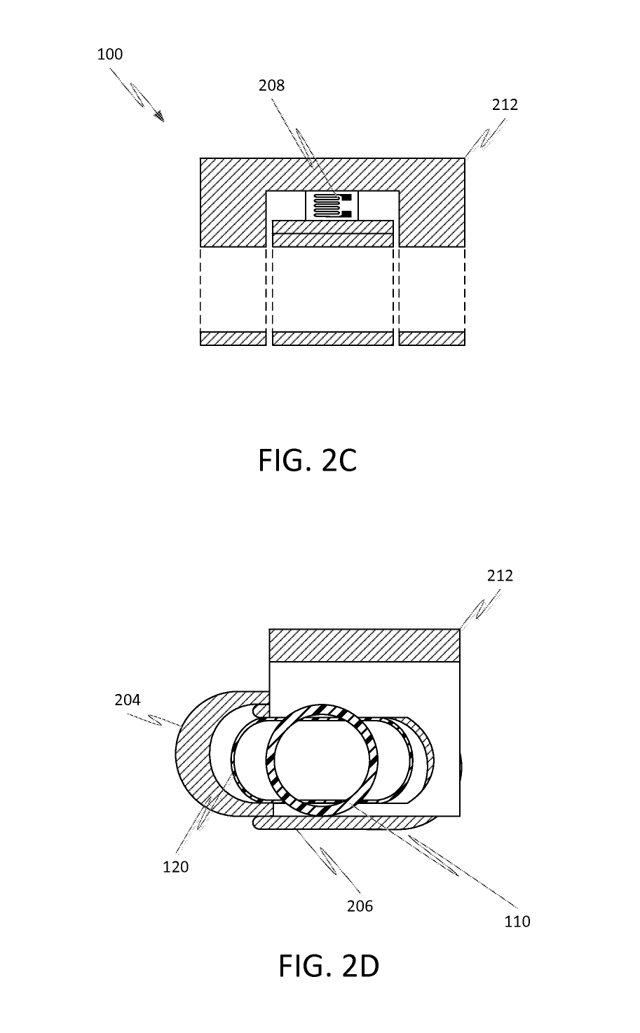

FIGS. 2A-2D show various views of a pressure sensing device 200 according to embodiments of the disclosed subject matter. Optionally, medical tubing 100 may be considered part of the pressure sensing device, in this embodiment or in any of the other embodiments. Device 200 includes an assembly of interleaved support members 204, 206 bonded (e.g., using an adhesive) to respective portions of the pillow portion structure 120 in an inline tube 100 or some other compliant conduit in order to perform negative pressure measurement. Such configuration can additionally hold the pillow portion 120 in an expanded state even under negative pressure. Further, when the flexible portion tries to collapse, the interleaved support members 204, 206 can be forced away from each other, with minimal displacement resulting from the forcing to cause compression of a pressure sensor 208 (e.g., a strain gauge) coupled to a stationary portion 212 on the force transducer 208 to provide a pressure signal corresponding to the negative pressure exerted by the fluid therewithin. Thus, pillow portion 122 can be caused to move by the interleaved support members 204, 206 and pillow portions 121 can be held stationary by corresponding parts of stationary portion 212. A clip 210 or alternatively recess walls in a blood treatment machine can be formed around the stationary portion 212 and support members 204, 206 as shown in FIGS. 2A and 2B to provide a rigid backing plate.

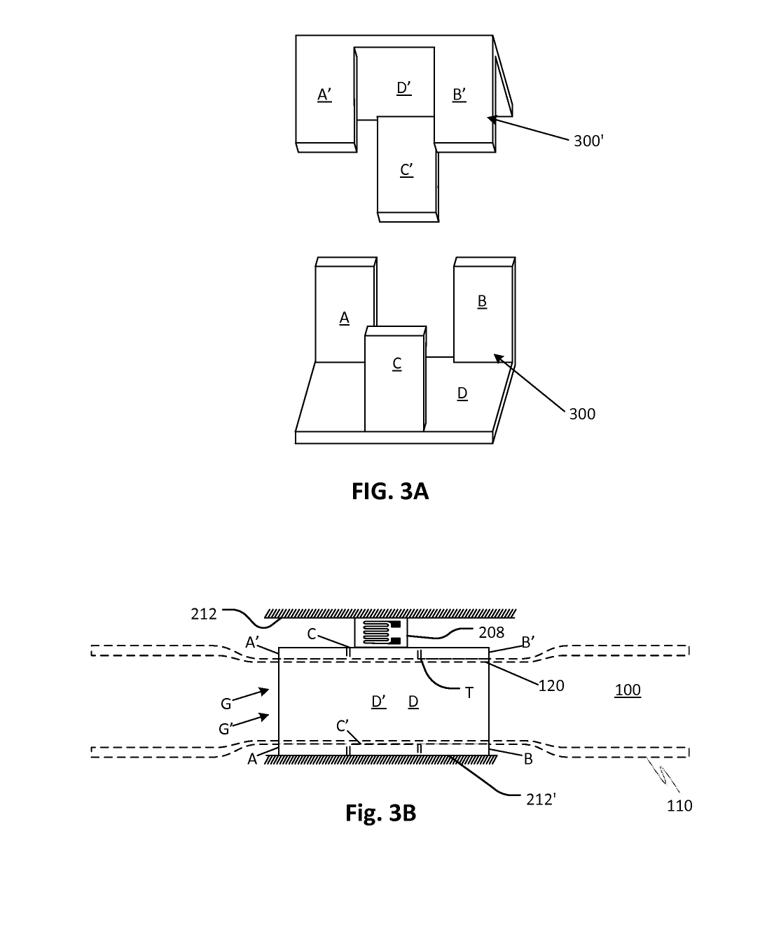

FIGS. 3A and 3B show a pair of interoperable members 300 and 300′ that may be adhesively attached to a pillow structure 120 in an inline tube 100 or some other compliant conduit to perform negative pressure measurement. First and second tines A and B respectively surround a tine C′ when member 300 is interleaved with part 300′. Similarly, First and second tines A′ and B′ respectively surround a tine C when member 300 is interleaved with part 300′. The interleaved arrangement is shown in FIG. 3B fitted in a rigid gap formed by opposing fixed walls 212 and 212′ with a force transducer 208 completing the first. The inner faces of the tines A, B, A′ and B′ are affixed to the wall of the pillow portion 120 so that when negative pressure within the pillow portion 120 pulls the attached tines inwardly, the tines C and C′ are forced away from each other, with minimal displacement resulting from the forcing and a force is exerted by tine C on the force transducer 208.

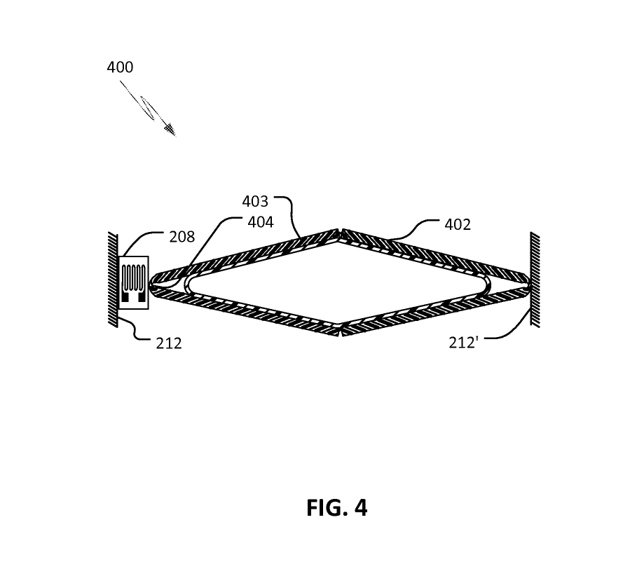

FIG. 4 shows pressure sensing device 400 having an arrangement with two V-shaped elements 402, 403 that have a living hinge 404 (could also be of metal or other materials and hinge could be a mechanical hinge or other equivalent) at the base of their V shapes so that they can flex easily. A pillow portion is adhesively bonded to the inner surfaces of the two V shaped elements 402, 403 and the device 400 is placed between two fixed walls 212 and 212′ with a force transducer 208 making up the gap between the two rigid walls 212, 212′. When negative pressure is applied to the lumen of the pillow portion, the V-shaped elements 402, 403 tend to collapse in the vertical direction forcing the hinge portions 404 outwardly thereby applying force to the force transducer.

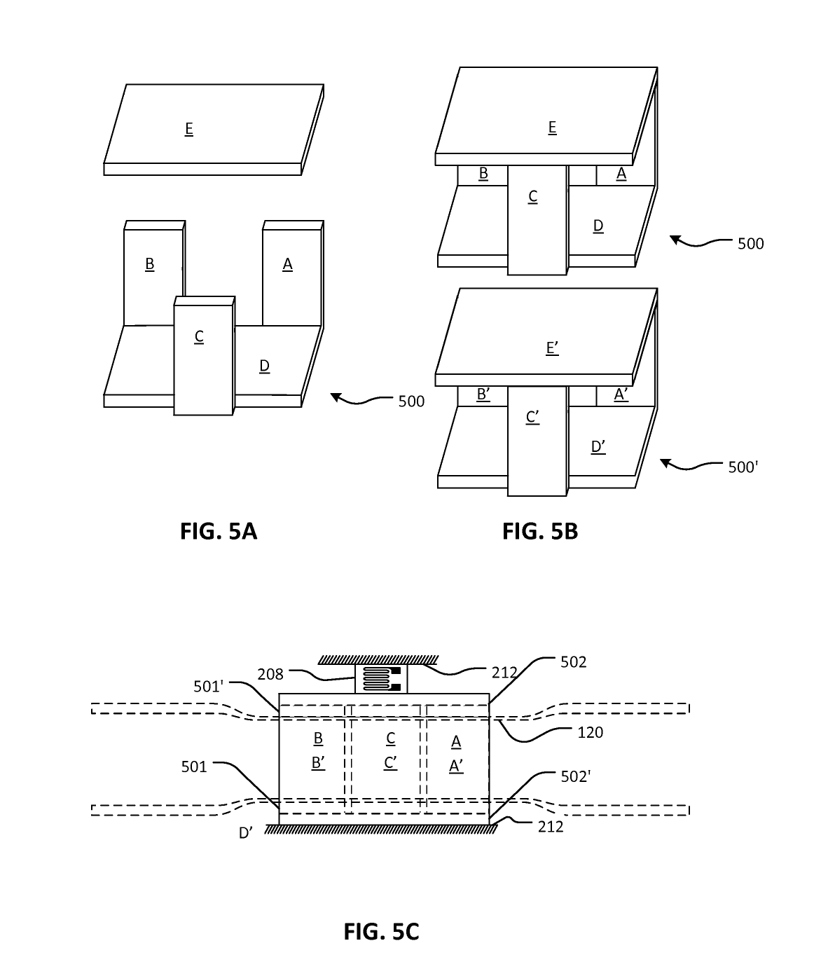

FIG. 5A shows a member 500 that is also configured to interleave with a like shaped member 500′. A pillow portion is adhesively bonded to the face of portion D and a facing plate E is attached to the tines A, B, and C to form a completed structure 500 as shown in FIG. 5B. The same structure is shown at 500′ with identically shaped features. Element 500 face D is bonded to the pillow portion. Then element 500′ face D′ is bonded to an opposite face of the pillow portion. Then the facing plate E and E′ are attached to tines A, B, and C of member 500 and to tines A′, B′, and C′ of member 500′ to form the completed structures 500 and 500′ in an interleaved arrangement shown in FIG. 5C.

In FIG. 5C, the assembled arrangement of the 500 and 500′ structures is fitted between facing rigid support faces 212 with a load cell 208 making up the gap. When the pillow portion 120 experiences negative pressure, the portions 501 and 501′ are pulled together and the portions 502 and 502′ are pulled apart. This applies a forced to the load cell 208 generating a signal.

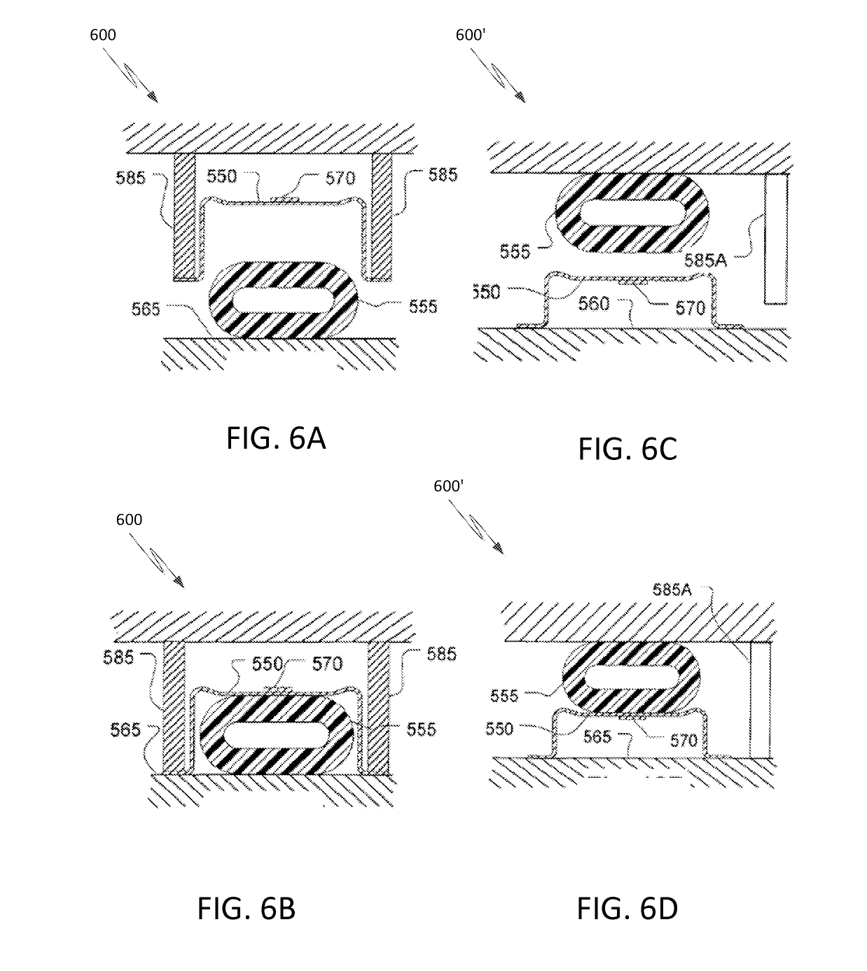

FIGS. 6A through 6D show pressure sensing devices 600 and 600′ according to embodiments of the disclosed subject matter. The devices 600 and 600′ shown in FIGS. 6A through 6D are based on the pressure sensors shown in FIGS. 13A through 13D of U.S. Pat. No. 7,337,674, the entire content of which is hereby incorporated by reference. As is apparent from FIGS. 6A through 6D, it is not a requirement that that the medical tubing has a thinned wall.

In FIGS. 6A and 6B, a flexible plate 550 is mounted between one or more standoffs 585. A strain gauge 570 is mounted on flexible plate 550, at a position where flexible plate 550 contacts the flattened portion of tube 555 when standoffs 585 are lowered to contact wall 565, as shown in FIG. 6B. Another aspect is shown in FIGS. 6C and 6D, in which tube 555 is mounted between standoffs 585, and a flexible plate 550 is mounted on wall 565. A strain gage 570 is mounted on flexible plate 550, and contacts the flattened portion of tube 555 when tube 555 is lowered so that standoff 585 contacts wall 565. Different from the embodiments in U.S. Pat. No. 7,337,674, the devices 600 and 600′ in FIGS. 6A through 6D have flexible plate 550 fixed to the tube 555, whereby when the tube flexes inward due to an internal negative pressure, the flexible plate 550 is caused to move with the tube 555 and thus the strain gauge 570 can output a corresponding signal representative of the internal pressure.

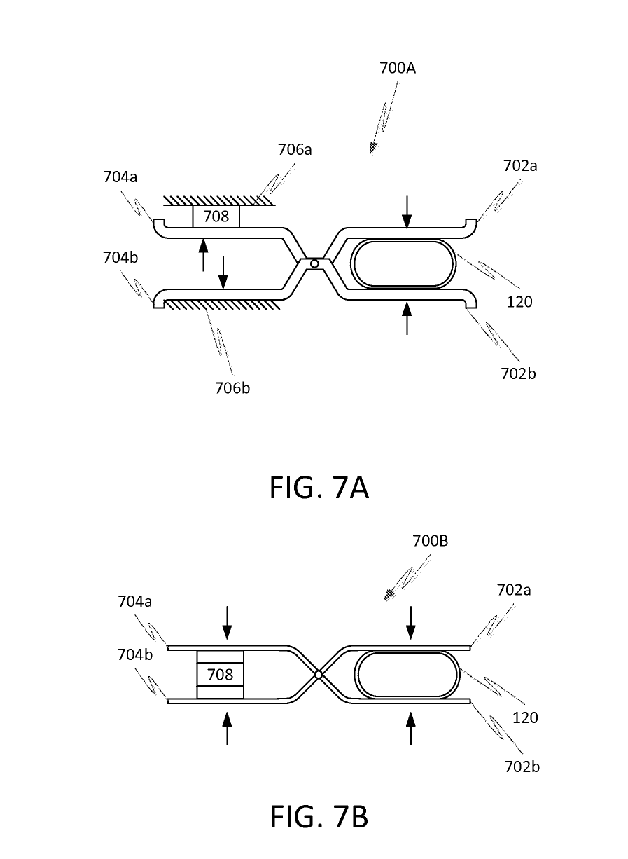

FIGS. 7A and 7B show pressure sensing devices 700A and 700B according to embodiments of the disclosed subject matter. Not explicitly shown, a flexible portion, such as pillow portion 120 can be adjacent to a portion of the devices 700A, 700B.

Device 700A is configured in a “scissor” configuration and can include portions 702 and 704, which can arms or plates, coupled, for instance, by an adhesive (e.g., glue) or mechanically by a flexible band or a string to opposite sides of medical tubing 100 adjacent the flexible or pillow portion 120 of the tubing 100. When internal forces act on pillow portion 120, the portions 702a and 702b are caused to move inwardly since they are adhered to the pillow portion 120, which causes corresponding movement of portions 704a and 704b as shown in FIG. 7A such that they act on transducer or load cell 708.

Device 700B is configured such that left and right bottom portions 702b and 704b are one piece and such that left and right top portions 702a and 704a are one piece. Portions 702 and 704 can be considered arms or plates, coupled, for instance, by an adhesive (e.g., glue) or mechanically by a flexible band or a string to opposite sides of medical tubing 100 adjacent the flexible or pillow portion 120 of the tubing 100. When internal forces act on pillow portion 120, the portions 702a and 702b are caused to move inwardly since they are adhered to the pillow portion 120, which causes corresponding movement of portions 704a and 704b as shown in FIG. 7B such that they act on transducer or load cell 708.

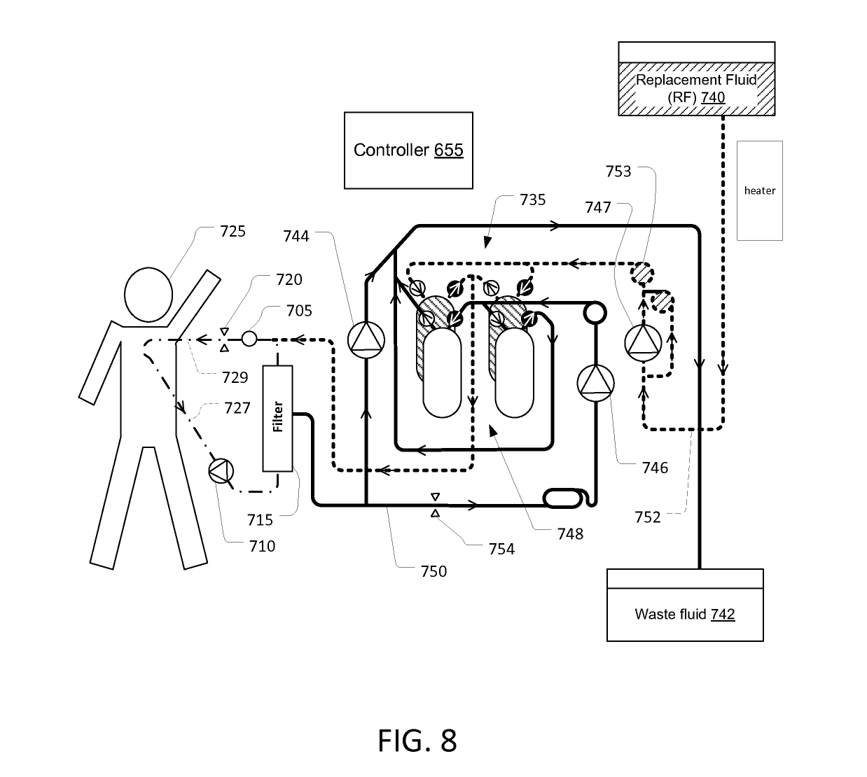

The use of the pressure sensor of the present invention in a blood treatment machine is illustrated schematically in FIG. 8, which corresponds to FIG. 16 in U.S. Pat. No. 7,337,674.

As indicated in U.S. Pat. No. 7,337,674, the operation of the blood treatment machine is described in detail in U.S. Pat. No. 6,638,478, which is hereby incorporated by reference in its entirety into the present application. Controller 655 regulates the flow rate of pumps 710, 744,746, and 747 to flow blood from the patient, through a blood processing device such as a hemofilter 715, and then back to the patient. Note that any kind of blood processing device or system may be employed, for example, a dialysis system and dialyzer, apheresis system and filter, adsorption blood cleansing regeneration system, etc. In the example, shown only for illustrating an application of the device, the machine includes a blood handling unit, a fluid management unit, and a ultrafiltration unit. The blood-handling unit circulates the patient's blood in a controlled manner through the hemofilter 715 and back to the patient after treatment. Note that the hemofilter 715 may be a dialyzer as well. The hemofilter 715 removes waste fluid, containing urea and other toxins, from the blood. The fluid management unit replaces the waste fluid with a sterile replacement fluid for return with the treated blood to the patient's blood supply. The replacement fluid also acts to maintain the patient's electrolytic balance and acid/base balance. The ultrafiltration unit removes waste fluid from the patient without the need for addition of replacement fluid.

Referring back to FIG. 8 herein, blood from the patient 725 is pumped by pump 710 through hemofilter 715 via arterial blood supply line 727, and then returned to the patient 725 via venous return line 729. Wastes, including liquid and uremic toxins, are separated by the hemofilter 715 from the rest of the blood.

The waste material exits the hemofilter 715 and is separated into an ultrafiltration path and a balancing path. Waste material in the ultrafiltration path is moved by pump 744 to a waste fluid container 742. Waste material in the balancing path is pumped by pump 746 through an inline balancing mechanism 749 that displaces replacement fluid, pumped by another pump 747, drawn from a replacement fluid chamber 740. Various valves, pumps and sensors are employed to determine and deliver the appropriate amount of replacement fluid required to insert into the venous return line to maintain the patient's blood pressure. The pressure sensor 705 shown in FIG. 8 can be representative of any of the pressure sensing devices according to embodiments of the present disclosure. Thus, placement of sensors according to embodiments of the disclosed subject matter can be placed at any suitable position in the arterial line.

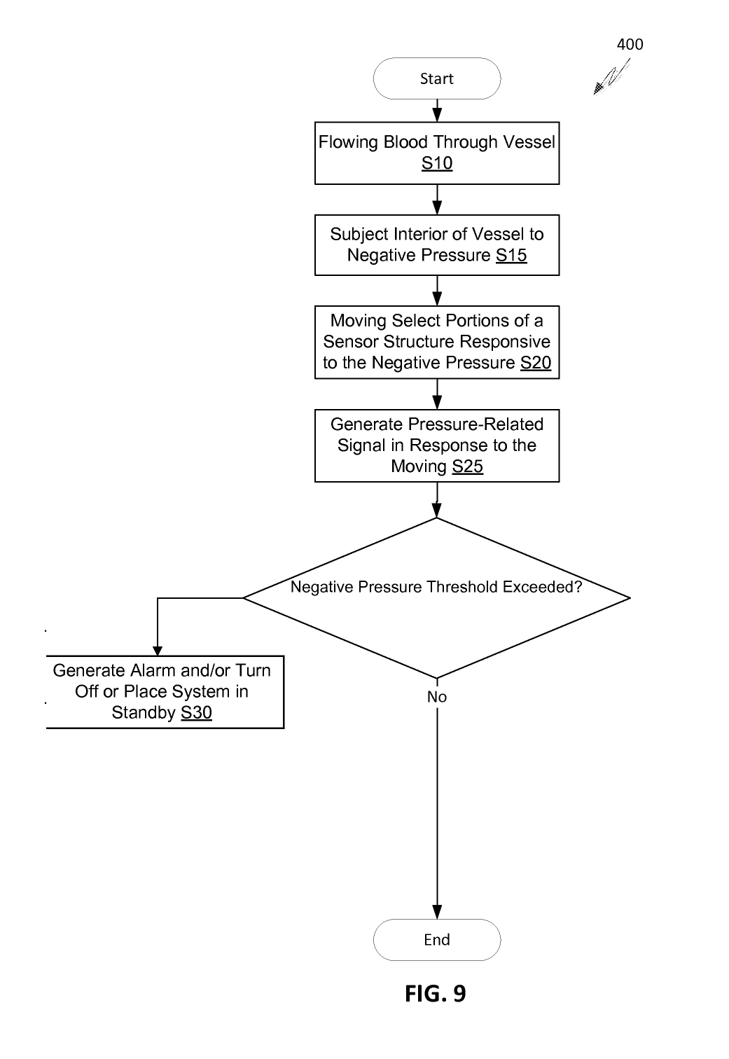

FIG. 9 is a flow chart for a method 900 according to embodiments of the disclosed subject matter.

Method 900 can be a method for negative pressure measurement or detection, whereby a signal corresponding to a pressure in a vessel can be generated. S10 represents a step of flowing fluid through a flexible vessel to which are attached a pair of members that are mutually movable. The flowing can subject an interior of the flexible vessel to a negative pressure (S15). The negative pressure can cause portions of a structure to move in response thereto (S20). For instance, the subjecting can be effective to move at least a portion of one member away from a portion of the other member so as to generate a progressively increasing separation therebetween. Responsive to the separating, a pressure signal can be generated including measuring a force of the progressively increasing separation (S25). Optionally, if the pressure signal indicates that the negative pressure has exceeded a threshold amount, one or more of the following is performed: activating an alarm and temporarily placing an associated fluid handling system in an off or standby state (S30).

Although embodiments described the use of attachment via adhesives, other attachment mechanisms may be employed, for example, fasteners, vacuum pumps, interference fits, snaps, Velcro, and other devices.

Having now described embodiments of the disclosed subject matter, it should be apparent to those skilled in the art that the foregoing is merely illustrative and not limiting, having been presented by way of example only. Thus, although particular configurations have been discussed herein, other configurations can also be employed. Numerous modifications and other embodiments (e.g., combinations, rearrangements, etc.) are enabled by the present disclosure and are within the scope of one of ordinary skill in the art and are contemplated as falling within the scope of the disclosed subject matter and any equivalents thereto. Features of the disclosed embodiments can be combined, rearranged, omitted, etc., within the scope of the invention to produce additional embodiments. Furthermore, certain features may sometimes be used to advantage without a corresponding use of other features. Accordingly, Applicant intends to embrace all such alternatives, modifications, equivalents, and variations that are within the spirit and scope of the present invention.

1. A negative pressure measurement device, comprising:
a fluid circuit portion for conducting a fluid in a portion of a blood circuit susceptible to negative pressure in support of a blood treatment therapy;
said fluid circuit portion having a tubular part with a sensing portion; and
a mechanism immediately adjacent the sensing portion of the tubular part that is configured and operative to translate a compliant strain of the fluid circuit portion responsive to a negative pressure therewithin into a force to the sensing portion to generate a signal;
wherein the force lies in a different principal direction from a principal direction associated with the strain.

2. The negative pressure measurement device according to claim 1, wherein said mechanism is configured and operative to prevent the sensing portion of the tubular part from collapsing or substantially collapsing due to internal negative pressure within the interior of the tubular part; portions of said mechanism being fixedly coupled to the sensing portion of the tubular part.

3. The negative pressure measurement device according to claim 1, wherein said mechanism includes an assembly of interleaved support members adhesively bonded to the sensing portion to hold the sensing portion in a fully expanded state or substantially fully expanded state even under negative pressure.

4. The negative pressure measurement device according to claim 3, wherein said mechanism is operative such that, when the sensing portion of said fluid circuit portion tries to collapse, the interleaved support members are operative on a strain gauge to provide a pressure signal corresponding to the negative pressure exerted by the fluid therewithin.

5. The negative pressure measurement device according to claim 3, wherein the interleaved support member is comprised of two support member portions each having a support panel and alternating prongs extending from respective first faces of the support panels; no portion of each of the prongs extending past the support panel of the other support member portion; prongs of both support member portions on first edges of the support panels being aligned and prongs of both support member portions on second edges of the support panels being aligned.

6. The negative pressure measurement device according to claim 1, wherein the sensing portion of said fluid circuit portion is part of a pillow portion thereof, the pillow portion being more flexible than immediately preceding and immediately following portions thereof.

7. The negative pressure measurement device according to claim 6, wherein the pillow portion is a product whose structure can be formed by heating a portion of a length of thermoplastic tubing and forcing a fluid into the heated portion to expand, and thin, the walls of the thermoplastic tubing or the pillow portion is a portion of a cylindrical tube whose walls are thinner than walls on either end thereof or the pillow portion is a non-cylindrical portion of a cylindrical tube.

8. The negative pressure measurement device according to claim 1, wherein at least the sensing portion of said fluid circuit portion is configured such that it suffers substantially no non-elastic strain in a wall thereof as a result of a pressure change within, said change in pressure including negative and positive pressure change, and whereby hysteresis in a signal from said sensor element is avoided or substantially avoided.

9. The negative pressure measurement device according to claim 1, wherein said mechanism has portions adhesively coupled to corresponding portions of the tubular part; a first adhered portion and a second adhered portion of the tubular part being prevented from moving inward due to negative pressure within the interior of the tubular part, and a third adhered portion of the tubular part corresponds to the sensing portion of the tubular part and is permitted to move very slightly in order for said mechanism to measure the negative pressure indication.

10. The negative pressure measurement device according to claim 1, wherein said mechanism includes a first support with a fixed surface positioned to support a fixed portion of the sensing portion and a pressure sensor to detect a pressure reading associated with deformation of a non-fixed portion of the sensing portion by detecting displacement thereof.

11. The negative pressure measurement device according to claim 10, wherein said pressure sensor includes a movable element with a movable surface substantially coplanar with said fixed surface.

12. A pressure measurement device for measuring pressure in a fluid-carrying tube portion of a disposable fluid circuit for a medical treatment system, comprising:
a sensor element positioned on a support of a fluid processing machine; said support being physically coupled to said tube portion when said fluid circuit is mounted on said fluid processing machine;
said sensor element being configured to generate a signal in response to a change in shape of said tube portion resulting from a negative change in pressure therewithin.

13. The device according to claim 12, wherein the support associated with said sensor element has at least one movable element with a movable surface that is affixed to said tube portion and at least two fixed elements that are affixed to said tube portion, each of said at least one movable elements is operative to move in response to the change in shape of said tube portion to thereby generate said signal.

14. The device according to claim 13, wherein said at least two fixed elements are operative to resist any movement in response to the change in shape of said tube portion.

15. The device according to claim 12, wherein said tube portion has a non-circular cross-section at a portion thereof associated with said sensor element.

16. The device according to claim 12, wherein said sensor element includes a strain detector.

17. The device according to claim 12, wherein the change in shape of said tube portion is minimal; said support preventing any significant narrowing of an internal volume of said tube portion due to the change in shape of said tube portion.

18. The device according to claim 12, wherein said tube portion suffers substantially no non-elastic strain in a wall thereof as a result of a change in pressure therewithin, whereby hysteresis in the signal from said sensor element is avoided.

19. The device according to claim 12, wherein the device is not configured and operative to measure or detect a positive change in pressure within said tube portion.

20-22. (canceled)

23. A method of generating a signal corresponding to a pressure in a vessel, comprising:
flowing fluid through a flexible vessel to which are attached a pair of members that are mutually movable;
subjecting an interior of the flexible vessel to a negative pressure thereby forcing the pair of members to move relative to each other;
the subjecting being effective to move at least a portion of one member away from a portion of the other member so as to generate a progressively increasing separation therebetween;
generating a pressure signal including measuring a force of the progressively increasing separation.
